(19) United States
(12) Patent Application Publication
(10) Pub. No.: US 2003/0196484 A1
(43) Pub. Date: Oct. 23, 2003

(54) Method to quantify total raw coalbed desorbed gas volume from geophysical logs

(76) Inventors: John Andrew Bowler III, Denver, CO (US); Steven Anthony Tedesco, Centennial, CO (US)

Correspondence Address: Bowler Petrophysics, Inc., Suite 835, 730 17th Street, Denver, CO, 80202, US

(21) Appl. No.: 10/336,703

(22) Filed: Jan. 3, 2003

Related U.S. Application Data
(60) Provisional application No. 60/373,667, filed on Apr. 18, 2002.

(51) Int. Cl. G01N 33/497

(52) U.S. Cl. 73/152.02; 73/19.01

(57) ABSTRACT

This method uses a simple relationship to compute total raw coal seam (coalbed) desorbed gas volumes from geophysical logs in a well bore. Laboratory total measured raw coal seam gas desorption volumes on coal core samples are calibrated to geophysical log measurements to obtain the relationship. The relationship has different constants and exponents for coal seams in each geologic basin and depositional setting. The calibrated relationship is applied to geophysical log measurements in cored and non-cored coal seams of the same type in the same basin to obtain a continuous total gas desorption volume estimate over the entire well in order to compute natural gas reserves in place. The method can be applied to coal seams where appropriate geophysical log measurements are available in any well.

### FIELD OF THE INVENTION

This invention relates to a use of geophysical logs to determine total raw coalbed gas desorbed from coal seams penetrated by a plurality of well bores.

### BACKGROUND OF THE INVENTION

This invention relates to the drilling of coalbed methane wells into the ground in order to intersect coal bearing formations and seams. Most coal seams contain methane adsorbed to the coal surfaces. Methane gas will desorb from the coals and enter the well bore after the well bore pressure is reduced. This gas can then be delivered from the well bore to the surface and sold as a natural gas energy supply. It is necessary to determine how much gas can be desorbed from the coals before proceeding with further expenditures. There must be enough methane gas adsorbed to the coals that can be desorbed for the operator to make a profit. This invention rapidly calculates desorbed gas volumes in place from geophysical logs recorded in well bores. Computer software yields the fastest results but calculations can be made by hand, calculator, computer spreadsheet, and by other means.

### SUMMARY

This invention minimizes the use of a lengthy and expensive laboratory desorption method to determine project viability and gas reserves calculation. This invention calculates continuous total raw gas desorption (gas volume)/(weight of coal) from geophysical logs after calibrating to laboratory derived desorption. This invention rapidly calculates gas reserves from geophysical logs which are already available. Calculations are very fast when this method is incorporated in a computer software program.

### BRIEF DESCRIPTION OF DRAWING

FIG. 1. is a logarithmic plot of total raw laboratory desorbed coal gas (labDES) against geophysical log bulk density (RHOB).

### DESCRIPTION OF THE PRIOR ART

A common means of determining total raw desorbed coalbed gas consists of:

1. Cutting a core in a coal seam during the drilling of the well.

2. Removing the core from the well and placing it in a sealed container (also known as a canister) at reservoir temperature.

3. Delivering the canister to the laboratory which is generally far from the well bore.

4. Allowing the gas to desorb in the laboratory at reservoir temperature over several months and measuring the desorbed gas volume from time to time.

5. Estimating lost and residual gas volumes and adding to desorbed gas volume to obtain (total desorbed gas volume)/(coal weight) in units of standard cubic feet of gas/ton of coal (scf/ton).

This process is costly. It can take several months before the results are known for the coal seams that have been cored and recovered from the well bore. Cores do not always contain coals and all coal seams in a well are not cored because it is too expensive to do so. This process does not measure desorbed gas volumes for coal seams that have not been cored and sent to the laboratory for testing.

No cores are cut in some well bores due to the expense. Very few coal seam cores are cut in well bores drilled for oil and gas.

Objects and Advantages

This method allows the accurate determination of gas reserves in place in non-cored and cored coal seams in a well and in adjacent or development wells without utilizing coring. This reduces costs and allows a gas company to increase revenue. This method also allows the calculation of gas in coal seams in wells drilled for conventional oil and gas reserves and water where before there was no method to do so without drilling a twin to the previously drilled well.

### DETAILED DESCRIPTION OF THE INVENTION

1. Do conventional laboratory analysis (not shown) of representative coal seam core samples throughout the coal seams of interest. Obtain laboratory measurements of total raw desorbed gas volume (labDES) in appropriate units such as standard cubic feet of gas/ton of coal (scf/ton). Select high, medium and low quality coal core samples. This is an important step to find a relationship between the geophysical logs and the raw coal seam gas desorption.

2. Enter the laboratory measurements in 1. into a spread sheet or an appropriate computer software program along with geophysical log in-situ measurements such as geophysical log bulk density (RHOB) value corresponding to the coal seam core sample. It may be necessary to depth match core depth to log depth so that RHOB measures the same coal recovered in the coal core. Coal samples may come from one or more wells.

TABLE 1
labDES and RHOB values in 23 coals from 2 wells in the same
geologic area.

| | | RHOB |
|---|---|---|
| | labDES | gram/cubic |
| Sample # | scf/ton | centimeter |
| 1. | 36.87 | 1.34 |
| 2. | 35.65 | 1.35 |
| 3. | 32.28 | 1.40 |
| 4. | 33.04 | 1.48 |
| 5. | 43.85 | 1.37 |
| 6. | 59.42 | 1.24 |
| 7. | 47.04 | 1.37 |
| 8. | 2.70 | 2.21 |
| 9. | 58.03 | 1.24 |
| 10. | 49.02 | 1.33 |
| 11. | 29.75 | 1.59 |
| 12. | 52.55 | 1.41 |
| 13. | 69.60 | 1.29 |
| 14. | 44.97 | 1.29 |
| 15. | 40.48 | 1.62 |
| 16. | 58.46 | 1.24 |
| 17. | 49.74 | 1.43 |
| 18. | 64.31 | 1.24 |
| 19. | 61.99 | 1.23 |
| 20. | 2.01 | 2.29 |
| 21. | 23.89 | 1.61 |
| 22. | 23.59 | 1.32 |
| 23. | 19.87 | 1.39 |

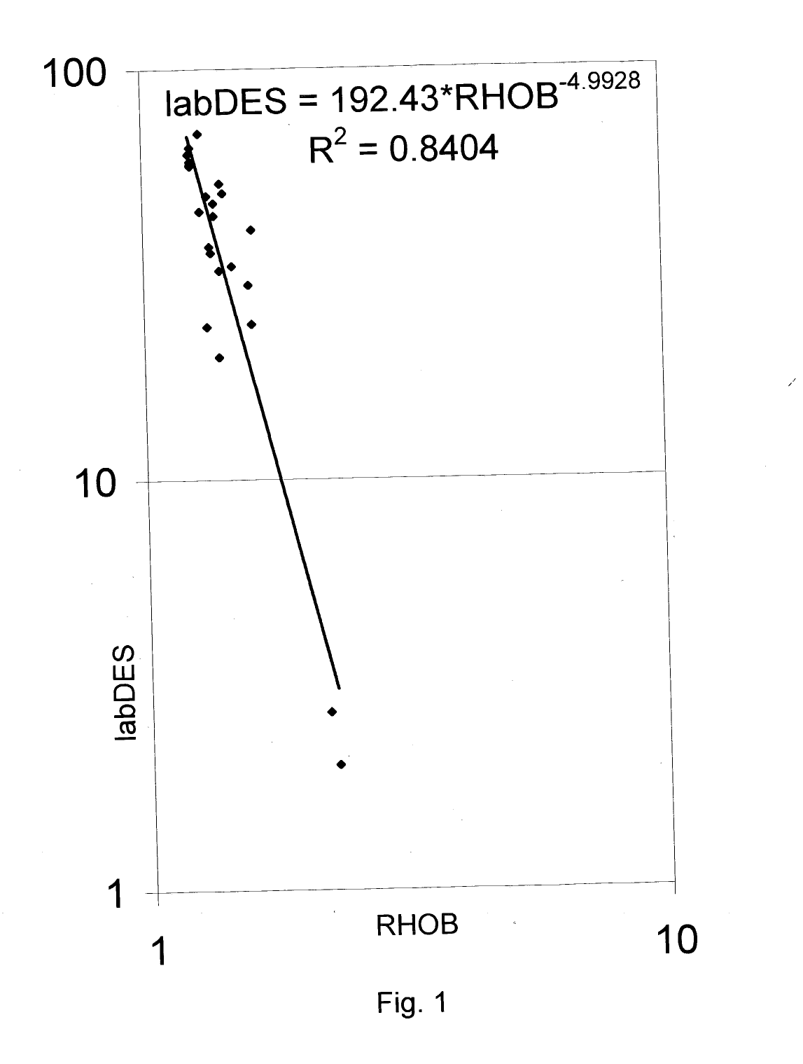

3. Plot total raw laboratory coal gas desorption (labDES) on the Y-axis (scf/ton) and plot RHOB on the x-axis (gram/cc). Try combinations of logarithmic and linear scales and curve fit labDES (Y-axis data) to RHOB (X-axis data). The best fit (data falls in a straight line) of Table 1 data is found in FIG. 1 using two logarithmic scales to obtain a power function: labDES=a*RHOB\hat{ }-b. labDES=192.43*RHOB\hat{ }−4.9928 with a −0.91675 correlation coefficient and R\hat{ }2=0.8404. Desorption increases when RHOB decreases.

The relationship is applied to RHOB in a spreadsheet or software program to calculate raw in-situ coal gas desorption (SCFdes) at every logged depth in the well bore. (SCFdes=192.43*RHOB\hat{ }−4.9928) is applied to coal seams in nearby wells that are geologically similar to those in the two wells where laboratory desorption (labDES) measurements have been made.

Gas is present in the higher density coaly shales and organic shales. If 4.0 scf.ton is the lower limit to be accepted for coalbed methane gas production it is possible to solve for the corresponding coal density.

Set labDES to 4.0 to solve labDES=a*RHOB\hat{ }b for RHOB to find the density value at which there is 4.0 scf/ton desorbed gas. This value can be used to determine a RHOB cutoff filter for the software program where no Net Pay gas is computed above the upper RHOB cutoff filter value.

4.0=192.43*RHOB\hat{ }−4.9928 RHOB=(192.43/4.0)\hat{ }−4.9928 RHOB=2.17

RHOB values below 2.17 gram/cc have enough gas desorption capability to be counted as a coalbed gas reservoir.

More than one geophysical log can be used to find a good relationship with desorbed gas. The product of two logs for example.

4. Load all of the digital geophysical log data as in Appendix A. into an appropriate computer software program or spreadsheet. JLog® commercial software is programmed to calculate desorbed coal bed methane gas reserve and is used in this example.

Enter the a constant and b exponent of the labDES=a*RHOBA\hat{ }-b power function determined in step 3. above in the software program. Cutoff filters are used to identify coal. Usually just one filter is needed. To detect coal with a RHOB cutoff filter: 1.10<RHOB<2.17 gram/cc. Geophysical log filter type and filter end points vary from well to well.

Enter drainage area which is the area in acres the well bore will drain of gas. This area depends on the distances between well bores and geology.

Gas in place calculations are now made by the computer software when RHOB falls below 2.17 gram/cc and above 1.10 gram/cc. If the well bore wall is washed out the RHOB measurement might read the mud density. 1.05 gram/cc for example. This is a faulty reading. With the RHOB cutoff filter set at 1.10 this washout will not be counted as Net Pay when estimating gas volumes.

The software program is now set up to compute Net Pay gas reserves at each depth recorded by the geophysical logs.

5. Enter the top and bottom depth of the well bore interval that is to be evaluated for coal bed methane gas Net Pay reserves.

The software reads in the depth and geophysical log values at each depth. If the cutoff filter conditions set by the user are met the following calculations are made (only RHOB cutoff filter set here) at each depth:

(a) SCFdes (Scf/ton)=a*RHOB\hat{ }-b.

(b) sub=depth increment*SCFdes*RHOB*(Drainage Area)*1,359.73. Where depth increment is the uniform depth increment at which the data is sampled by the geophysical log. 0.25 feet depth increment in this example. When the depth increment is not regular the difference between the previous depth and the present depth is used for depth increment. The 1,359.73 conversion factor converts units to standard cubic feet of gas at surface conditions.

(c) Add this sub value to the previous sum of sub values to keep a running sub total called subsum. At each level: subsum=subsum+sub.

(d) Increase the Net Pay footage by the depth increment.

(e) Present the Net Pay feet.

(f) Compute and show thickness and desorbed gas volume of each coal.

(g) Present computed results in tables of text on the screen and in text and LAS ASCII files saved to disc. See Appendix B. for results of these calculations and calculated total desorbed gas contained in coal seams represented by data in Appendix A.

(h) Present computed results in graphical form (not shown) on screen and in graphics files (not shown) saved to disc.

(i) Print the tables as in Appendix B. and graphics of computed results on a color printer (not shown).

Method to Quantify Total Raw Coalbed Desorbed Gas Volume from Geophysical Logs

We claim:

1. A method to quantify total raw coalbed desorbed gas volume in subterranean coal seams by:
(a) determining a relationship between laboratory measured total desorbed coal seam gas volume and coal seam geophysical log measurements in a well bore;
(b) applying said relationship to said geophysical log measurements to quantify a total raw coalbed desorbed gas volume.

2. The method in claim 1 is applied to coal seams where the geophysical log measurements are available in any well whether it has been drilled for coal seam natural gas, water, oil, natural gas, or another reason and whether or not the laboratory desorption measurement has been made on any of the coal seams.

3. The method in claim 1 reduces the amount of coal seam coring and laboratory desorption testing needed to evaluate the coal seam gas reserves in a well or in any number of wells.
